(19) United States
(12) Patent Application Publication
(10) Pub. No.: US 2009/0124934 A1
(43) Pub. Date: May 14, 2009

(54) GUIDEWIRE TORQUE DEVICE

(75) Inventors: Gerard Rabbitte, Tuam (IE); David Vale, Palo Alto, CA (US)

Correspondence Address: WORKMAN NYDEGGER, 1000 EAGLE GATE TOWER,, 60 EAST SOUTH TEMPLE, SALT LAKE CITY, UT, 84111, US

(73) Assignee: ABBOTT LABORATORIES, Abbott Park, IL (US)

(21) Appl. No.: 11/938,028

(22) Filed: Nov. 9, 2007

(51) Int. Cl. A61M 25/00; A61B 5/00

(52) U.S. Cl. 600/585

(57) ABSTRACT

In accordance with the present invention there is provided a guidewire torquing device configured to be readily attachable and detachable from a guidewire.

### BACKGROUND OF THE INVENTION

1. The Field of the Invention

The present invention relates generally to apparatus and methods for steering medical devices, and more particularly for steering guidewires.

2. The Relevant Technology

Interventional medical procedures involving guidewire introduction, insertion and manipulation are well known. For example, angioplasty generally includes the steps of inserting a guidewire through a vascular access needle into the femoral artery, and manipulating the external proximal end of the guidewire to advance the distal end of the guidewire through the patient's arterial tree to a predetermined vascular destination. A conventional dilatation catheter, or other percutaneous catheter, is then easily and rapidly fed over the guidewire directly into the vasculature to perform an appropriate diagnostic or therapeutic procedure. Similar guidewire applications are utilized in the introduction, removal and/or exchange of various catheters and like apparatus.

Insertion and manipulation of guidewires is often difficult and time consuming. Handling of guidewires is further complicated by the fact that conventional guidewires are generally provided with protective coatings that prevent blood from clotting on the guidewire, improve biocompatibility and enhance the guidewires maneuverability through the vascular system. However, such coatings often become more slippery when wetted, particularly with blood.

Still further, embolic protection devices such as those shown and disclosed in US Patent (Emboshield) and US Patent (Accuent) have filtering devices mounted to or disposed upon guidewires. These devices are generally inserted distal to a region to be treated in the carotid artery. In order to advance these devices, the physician must navigate tortuous anatomy with the guidewire by torquing the guidewire. Further still, a guidewire torquing device may be utilized to load the embolic protection filter into a delivery catheter. Conventional torquing devices generally require that the guidewire be disposed through a lumen of the torquing device or disposed within an aligned slot. The user then must hold onto a portion of the device while tightening a second portion of the device about the guidewire. If the user wishes to move the torquing device along the length of the guidewire, then they must utilize two hands in order to move the device or request additional assistance.

As such, a need exists for a user friendly guidewire steering device providing physicians with a convenient and reliable tool for handling guidewires. The present invention provides such a steering device.

### BRIEF SUMMARY OF THE INVENTION

In accordance with the present invention there is provided a guidewire torquing device configured to be readily attachable and detachable from a guidewire.

In accordance with the present invention there is provided a guidewire torquing device having a housing, the housing having a proximal end, a distal end and a lumen disposed therethrough. A proximal end cap is associated with the proximal end of the body and a distal end cap is associated with the distal end of the body, the distal end cap having an inner taper surface. A collet is disposed within the lumen of the body, the collet having a proximal end a distal end, an aperture and at least one groove formed partially therebetween. The distal end of the collet has a tapered surface, the tapered surface of the collect being shaped having a taper complementary to that of the inner surface of the cap. The device further includes a lever pivotally associated with the body and an actuator associated with the lever, the actuator being configured to cause the collet to engage and disengage a guidewire disposed through the aperture thereof.

In accordance with the present invention there is provided a method of attaching a torquing device to a guidewire. The method including the steps of (1) providing a guidewire torquing device, the guidewire torquing device having a lumen disposed therethrough, the lumen configured to receive a guidewire therein; (2) disposing a guidewire through the lumen; (3) moving a lever associated with the guidewire torquing device from a first position to a second position, wherein movement of the lever causes a force to be applied to a locking collet disposed within the torquing device, the locking collet moving from a first position to a second position, whereby the torquing device engages the guidewire.

These and other objects and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 is an isometric view of an exemplary embodiment of a torquing device in accordance with the present invention;

FIG. 2 is a cross-sectional plan view of the torquing device of FIG. 1;

FIG. 3 is a cross-sectional plan view of the torquing device of FIG. 1, illustrating the torquing device in a first state;

FIG. 4 is a cross-sectional plan view of the torquing device of FIG. 1, illustrating the torquing device in a second state; and

FIG. 5 is a side view of a guidewire usable with the torquing device of FIG. 1.

It should be noted that the figures are not drawn to scale and that elements of similar structures or functions are generally represented by like reference numerals for illustrative purposes throughout the figures. It also should be noted that the figures are only intended to facilitate the description of the illustrated embodiments of the present invention. The figures do not describe every aspect of the present invention and do not limit the scope of the invention.

### DETAILED DESCRIPTION

Generally, the present invention is directed toward an apparatus and method for steering medical devices. In one configuration, the apparatus and method can be associated with a guidewire steering device, although the apparatus and method can have applicability to other medical devices. With respect to the exemplary configuration, the apparatus can be configured to receive and retain a guidewire of any size. The torquing device of the present invention is also configured to be used single-handed.

Accordingly, there is provided a guidewire torquing device. The torquing device includes a body having proximal and distal ends, a distal cap disposed at the distal end, an end cap disposed at the proximal end, a collet disposed within a lumen of the distal cap and an actuator associated with the distal cap and the collet.

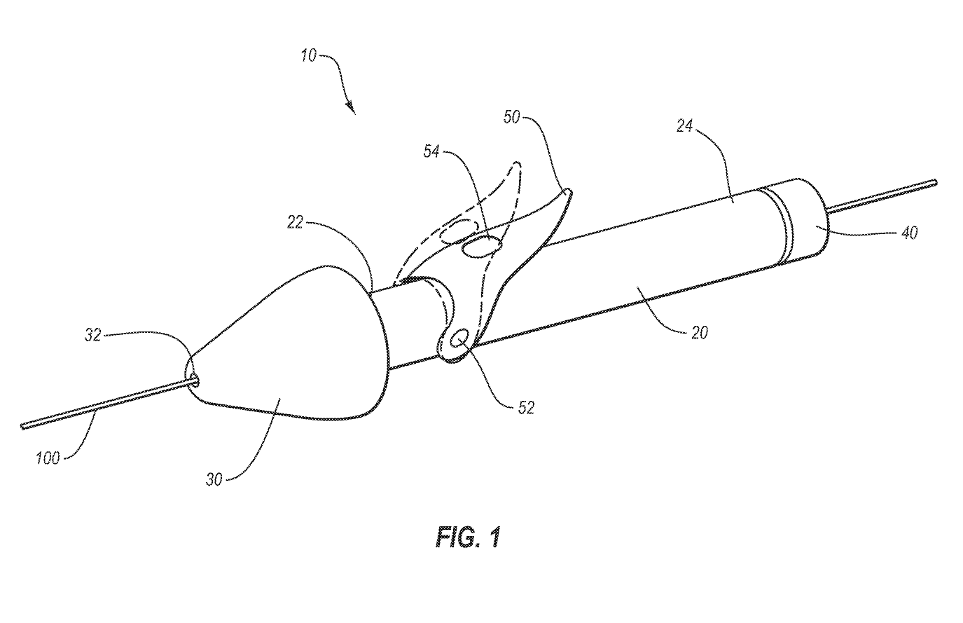

Referring now to FIG. 1 there is shown a perspective view of an exemplary embodiment of the torquing device in accordance with the present invention. As shown in FIG. 1, the torquing device 10 includes a body 20 having a distal end 22 and a proximal end 24, a cap 30 disposed about and adjacent to the distal end 22 of the body 20 and a proximal end cap 40 disposed at the proximal end 24 of the body 20. The torquing device 10 further includes a lever 50 pivotally attached through pivot 52 to a portion of the body 20, the lever 50 cooperating with an actuator or actuator assembly that causes the torquing device 10 to engage or disengage with a guidewire 100 disposed through portions of the torquing device 10.

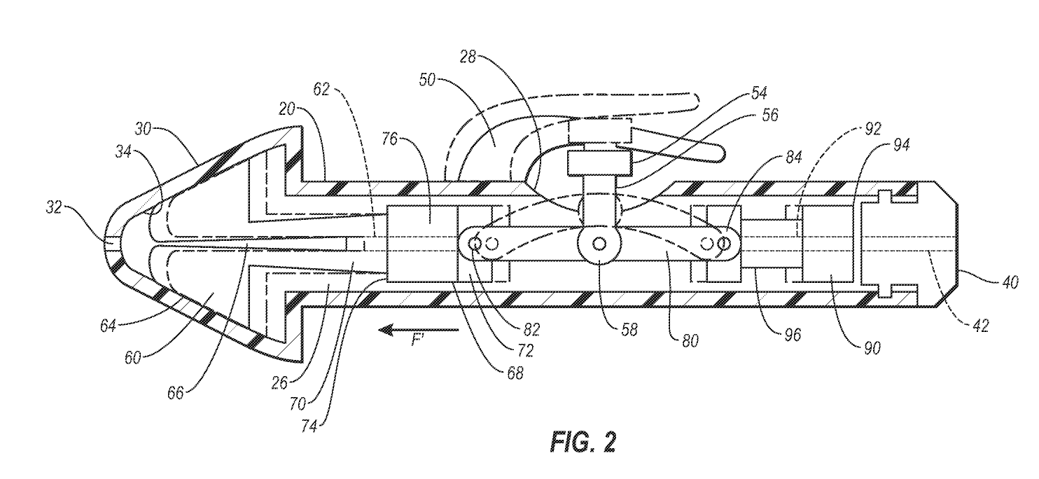

Referring now to FIG. 2 there is shown a cross-sectional view of the torquing device 10 in accordance with the present invention. As shown in FIG. 2, cap 30 is configured to retain a collet 60 within a lumen 26 of the body 20. Cap 30 may be threadably attached to the body 20 to retain the collet 60 or may be affixed to the body 20 using suitable attachment means such as, but not limited to, glues, laser welding, bonding or other known attachment methods. Alternatively, the cap 30 may be integrally formed with the body 20 such that the body and cap are formed as a single unit.

With continued reference to FIG. 2, collet 60 includes a collet lumen 62 formed therethrough, the collet lumen 62 being sized to receive a medical device, such as a guidewire or other medical devices of various size therethrough and being axially aligned with a lumen 32 of the cap 30. Collet 60 further includes a tapered distal portion 64; the tapered distal portion or surface 64 of the collet 32 is received by a tapered inner surface 34 of the cap 30 as shown in FIG. 2. Collet 60 further includes at least one slot 66 formed therein as shown in FIG. 2, the slot 66 is formed having sufficient size and shape to allow a portion of the collet 60 to be reduced in diameter as will be described in detail below with regard to the methods of use of the present invention.

Disposed adjacent to a proximal end 70 of the collet 60 is a collet ring 68, the collet ring 68 is sized to be slidably received within the lumen 26 of the body 20 and includes a proximal end 72, a distal end 74, and a collet ring lumen 76 extending from the proximal end 72 towards the distal end 74. The distal end 74 of the collet ring 68 is sized to abut the proximal end 70 of the collet 60 as shown, while the collet ring lumen 76 is axially aligned with the lumen 32 of the cap 30 and the collet lumen 62 of the collet 60 to receive guidewires or other medical devices of various sizes.

The proximal end 72 of the collet ring 68 is coupled to a distal end 82 of a spring element 80. The spring element 80 has the distal end 82 coupled to the collet ring 68 and a proximal end 84 coupled to an end cap support 90. The end cap support 90 is sized and configured to be slidably received within the lumen 26 of the body 20 and includes a support lumen 92 extending from the proximal end 94 towards the distal end 96. This support lumen 92 is axially aligned with the lumens 32, 62, and 76 to receive guidewires and medical devices of various sizes.

In addition to being coupled to the end cap support 90, the spring element 80 is coupled to a shaft 54. A distal end 58 of a shaft 54 is coupled to the spring element 80 between the proximal end 84 and distal end 82 thereof. The proximal end 56 of the shaft 54 is configured to project through an aperture 28 formed in the body 20, wherein the proximal end 56 of the shaft 54 projects beyond an outer surface of the body 20.

As shown in FIG. 2, the spring element 80 is configured to translate from a first position (shown in dotted outline) to a second position and can form part of the actuator or actuator assembly which is configured to cause the collet 60 to engage and disengage the guidewire 100. The spring element 80 is translatable between the first and second position though application of force to the shaft 52. The lever 50 as shown in FIG. 1 and shown in FIG. 2 in dotted outline is rotationally attached to the body 20, wherein a portion of the lever 50 is configured to interface with the proximal end 56 of the shaft 54. The lever 50 is configured to be movable between a first position and a second position, thereby cooperating with the associated actuator or actuator assembly that causes the collet to engage and disengage a guidewire. The actuator or actuator assembly can include one or more of the shaft 54, the collet ring 68, the spring element 80, and/or the end cap support 90. Further, the actuator or actuator assembly can optionally include the lever 50.

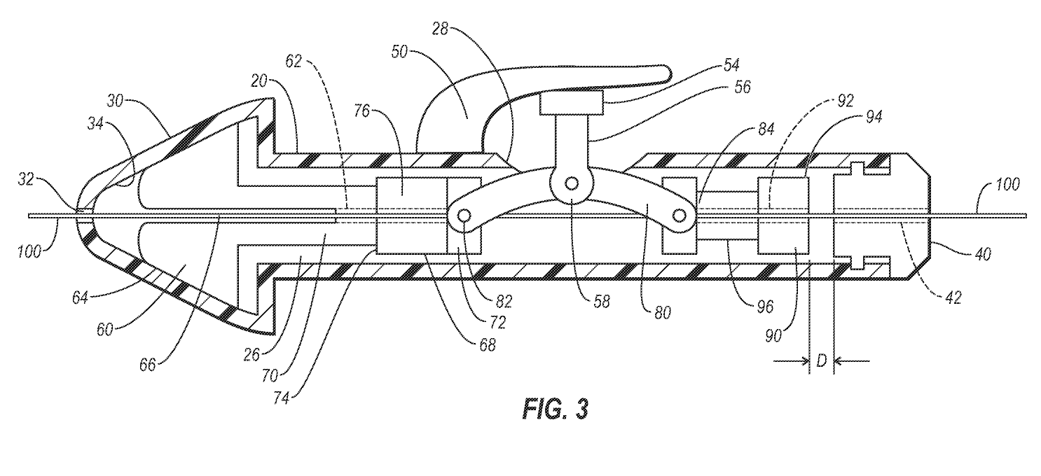

Referring now to FIG. 3 there is shown a cross-sectional view of the torquing device of the present invention, wherein a guidewire 100 is shown disposed through a passageway or lumen formed by the lumen 32 of the cap 30, the collet lumen 62 of the collet, the collet ring lumen 76 of the collet ring 68, the support lumen 92 of the end cap support 90, and a lumen 42 of the cap 40. As shown in FIG. 3, the lever 50 is disposed in the first position, wherein no force F is being applied to the proximal end 54 of the shaft 52. In this configuration, the torquing device can be freely translated and rotated along the length of the guidewire 100.

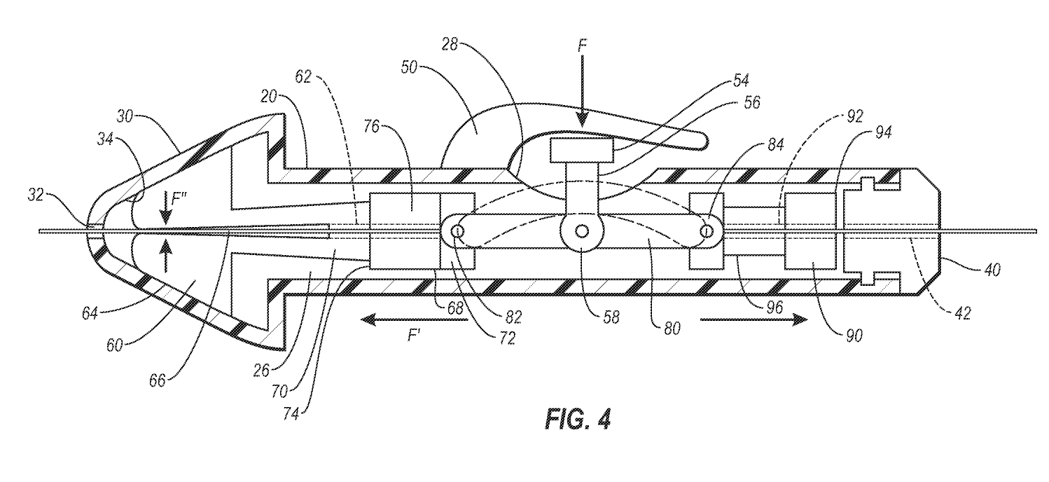

Referring now to FIG. 4, there is shown the torquing device 10 of the present invention wherein the lever 50 has been moved from a first position to a second position. When lever 50 is disposed in the second position, a force F is applied to the proximal end 54 of the shaft 52, as a result of the applied force F, shaft 52 is translated along an axis perpendicular to the lumen 32, whereby the force applied to the shaft 52 is translated through the spring element 80, thereby moving the spring element 80 from a first position, whereby the spring element 80 is in a relaxed state, to a second position, whereby the spring element 80 is a compression state. The force F applied to the shaft 52 is translated by spring element 80 into a force F′ along the axis of the guidewire 100. Additionally, movement of the spring element 80 from the first position to the second position causes translation of the end cap support 90 and the collet ring 68 within the lumen 26 of the body 20. The end cap 40 limits the translation of the end cap support 90 or limits the distance of travel of the end cap support 90 within the lumen 26 of the body 20. This limited travel also aids with the translation of the collet ring 68 within the lumen 26. The translation of the collet ring 68 within the lumen 26 of the body 20 causes a linear force to be applied to the collet 60, thereby causing the collet 60 to translate within the lumen 26 and within the cap 30.

As described above, the collet 60 includes a tapered surface 64. The tapered surface 64 is received by a complementary tapered surface 34 of the cap 30 as the collet 60 is translated within the lumen 26 of the body 20. The collet 60 is forced against the tapered surface 34 of the cap 30, thereby causing the groove 66 of the collet 60 to be reduced in size and causing the collet 60 to grip the guidewire 100 disposed within the lumen 32 with a force F″. The force F″ can be adjusted by changing the resting distance D, as shown in FIG. 3, between the end cap 40 and the end cap support ring 90. The distance D can be adjusted by threading the end cap 40 into and out of the lumen 26 of the body 20. By providing the adjusting feature, the user can adjust the amount of force F″ applied to the guidewire 100. Additionally, the torquing device 10 of the present invention can be adjusted for various diameter guidewires or medical devices.

It will be understood that the application and release of the forces F, F′, and F″ may be repeated many times on the same guidewire or medical device in a single-handed procedure. For instance, a user of the torquing device of the present invention can single-handedly position the torquing device upon the guidewire or medical device through moving the lever to activate the actuator or actuator assembly so as to engage the guidewire or medical device and subsequently single-handedly release the lever to release or disengage the actuator or actuator assembly and consequently release or disengage the guidewire or medical device. Once released the user can reposition the torquing device and repeat the process or steps to re-engage the guidewire or medical device.

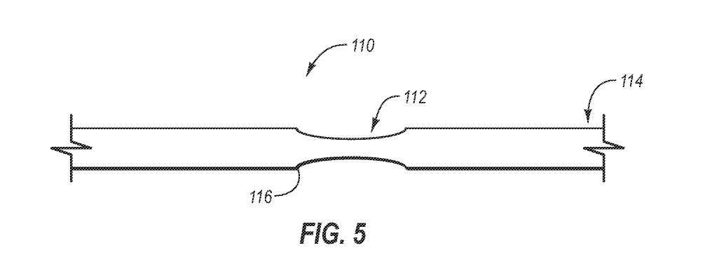

While the torquing device 10 of the present invention can be utilized with convention guidewires, it may be desirable to provide a guidewire having a reduced diameter portion disposed adjacent to the proximal end. Referring now to FIG. 5, there is shown an exemplary embodiment of a guidewire in accordance with the present invention. As illustrated, the guidewire 110 includes a reduced diameter portion 112 disposed adjacent to the proximal end 114. The reduced diameter portion 112 allows the collet 60 (FIG. 4) to grip the portion of the guidewire 110 in a more secure fashion without causing damage to the guidewire 110. The reduced diameter portion 112 further provides a shoulder 116 on which the collet 60 of the torquing device 10 can abut when the torquing device 10 is activated to become locked to the guidewire 110. This shoulder 116 is useful if a linear force needs to be applied to the guidewire 110; the collet 60 will abut the shoulder 116 and be prevented from slipping along the length of the guidewire 110. Additionally, the reduced diameter portion 112 may be free of any coatings that may be disposed along the length of the guidewire 110. Fore example, the guidewire 110 may be coated with a hydrophilic coating or other lubricious or slippery coating.

The torquing device in accordance with the present invention may be utilized in various medical procedures, such as stenting, angioplasty, atherectomy, carotid stenting, embolic protection filter deployment/retrieval, drug delivery and other therapies that require the use of a torquing device. It shall be further understood that although the present invention has been described for use with guidewires, it shall be understood that the device can be utilized with other medical devices where it is desired to either impart a torquing force or provide a removable handle thereon.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A guidewire torquing device, comprising:
a body having a proximal end, a distal end and a lumen disposed therethrough;
a proximal end cap associated with the proximal end of the body;
a distal end cap associated with the distal end of the body, the distal end cap having a tapered inner surface;
a collet disposed within the lumen of the body, the collet having a proximal end, a distal end, a collet lumen and at least one groove formed partially therebetween, the distal end of the collet having a tapered surface, the tapered surface of the collect being shaped having a taper complementary to that of the inner surface of the cap;
a lever pivotally associated with the body; and
an actuator associated with the lever, the actuator configured to move the collet to engage and disengage a guidewire disposed through the collet lumen thereof, the lever being pivotal towards the body to engage the collet with the guidewire and pivotal away from the body to disengage the collet with the guidewire and secure the guidewire torguing device to the guidewire.

2. The torquing device according to claim 1, wherein the proximal end cap is threadably engaged with the body

3. The torquing device according to claim 1, wherein the actuator comprises a spring element and a shaft, the shaft cooperating with the lever to move the spring element.

4. The torquing device according to claim 1, wherein the actuator further comprises at least one of a collet ring cooperating with the collet and an end cap support cooperating with the proximal end cap.

5. The torquing device according to claim 4, wherein the proximal end cap limits movement of the end cap support within the lumen of the body.

6. A guidewire torquing device, comprising:
a body having a distal end cap, a proximal end, and a lumen extending from the distal end cap towards the proximal end, the distal end cap having a tapered inner surface;
a collet disposed within the lumen of the body, the collet having a tapered distal end complementary to the tapered inner surface of the body, a proximal end, and at least one groove extending from the distal end toward the proximal end; and
an actuator assembly associated with the body, a first portion of the actuator assembly being pivotally movable relative to the body and a second portion of the actuator assembly being slidably movable within the body toward the proximal end and the distal end of the body to selectively engage the collet with a guidewire disposed through the collet lumen.

7. The torquing device according to claim 6, further comprising a proximal end cap threadably coupled with the body

8. The torquing device according to claim 7, wherein the actuator assembly comprises a lever pivotally coupled to the body and a spring element slidably movable within the lumen of the body, the lever and the spring element being linked by a shaft.

9. The torquing device according to claim 8, wherein the actuator assembly further comprises at least one of a collet ring cooperating with the collet and an end cap support cooperating with the proximal end cap.

10. The torquing device according to claim 9, wherein the proximal end cap limits movement of the end cap support within the lumen of the body.

11. A method of attaching a torquing device to a guidewire, comprising
providing a guidewire torquing device, the guidewire torquing device having a body and a lumen disposed therethrough, the lumen configured to receive a guidewire therein;
disposing a guidewire through the lumen; and
moving a lever associated with the guidewire torquing device from a first position with a portion of the lever being spaced apart from the body to a second position with the portion of the lever being moved toward the body, wherein movement of the lever applies a force to a locking collet disposed within the torquing device, the locking collet moving from a first position to a second position to engage; the torquing device to the guidewire.

12. The method as recited in claim 11, wherein the guidewire torquing device further comprises a distal end cap with a distal end cap lumen, and a proximal cap having a proximal cap lumen and wherein disposing a guidewire through the lumen further comprises locating the guidewire within the lumen, the distal end cap lumen, and the proximal cap lumen.

13. The method as recited in claim 12, wherein the guidewire torquing device further comprises a collet with a collet lumen, a collect ring with a collet ring lumen, and an end cap support with a support lumen and where disposing guidewire through the lumen further comprises disposing the guidewire through the collet lumen, the collet ring lumen, and the support lumen, the guidewire having a shoulder to be received by the guidewire torquing device.

14. The method as recited in claim 11, further comprising moving the lever from the second position to the first position to release the force applied to the locking collet to disengage the guidewire torquing device form the guidewire.

15. The method as recited in claim 14, further comprising repositioning the guidewire torquing device relative to the guidewire and moving the lever associated with the guidewire torquing device from the first position to the second position to engages the guidewire torquing device with the guidewire.

16. A method of selectively positioning a torquing device to a medical device, the method comprising
positioning a torquing device upon a medical device, the torquing device having a body and a lumen therethrough and a locking collet disposed therein, the lumen and the locking collet each configured to receive a portion of the medical device; and
moving a lever associated with the torquing device from a first lever position with a portion of the lever being spaced apart from the body to a second lever position with the portion of the lever being moved toward the body, movement of the lever causing a force to be applied to the locking collet to move the locking collet from a first collet position allowing the torquing device to move along the medical device to a second collet position whereby the torquing device engages the medical device and the torquing device is prevented from moving along the medical device.

17. The method according to claim 16, wherein positioning the torquing device upon the medical device further comprises slidably receiving the medical device within the lumen of the torquing device.

18. The method according to claim 16, wherein moving the lever comprises pivotally moving the lever coupled to the body of the torquing device.

19. The method according to claim 16, further comprising single-handedly moving the torquing device along a portion of the medical device and moving the lever associated with the torquing device to engage the torquing device with the medical device.

20. The method according to claim 19, further comprising single-handedly releasing the lever to disengage the torquing device from the medical device to selectively reposition the torquing device.
